(19) United States
(12) Patent Application Publication
(10) Pub. No.: US 2021/0272694 A1
(43) Pub. Date: Sep. 2, 2021

(54) PREDICTING BIOCHEMICAL RECURRENCE BASED ON COMPUTERIZED QUANTIFICATION OF CRIBRIFORM MORPHOLOGY

(71) Applicant: Case Western Reserve University, Cleveland, OH (US)

(72) Inventors: Anant Madabhushi, Shaker Heights, OH (US); Sacheth Chandramouli, Chicago, IL (US); Patrick Leo, Honeoye Falls, NY (US); Andrew Janowczyk, East Meadow, NY (US)

(21) Appl. No.: 17/140,544

(22) Filed: Jan. 4, 2021

Related U.S. Application Data
(60) Provisional application No. 62/983,939, filed on Mar. 2, 2020.

(51) Int. Cl. G16H 50/20; G16C 20/70; G16B 40/20; G06N 3/08; G06T 7/00

(52) U.S. Cl. CPC G16H 50/20; G16C 20/70; G16B 40/20; G06T 2207/20081; G06T 7/0012; G06T 2207/30096; G06T 2207/20084; G06N 3/08

(57) ABSTRACT

Embodiments discussed herein facilitate determination of a likelihood of biochemical recurrence (BCR) of cancer (e.g., prostate cancer, etc.). One example embodiment is a method, comprising: accessing at least a portion of a digitized stained histology slide comprising a tumor; automatically segmenting, via a trained deep learning (DL) model, cribriform morphology in connection with the tumor on the at least the portion of the digitized stained histology slide; determining a cribriform-to-tumor area ratio (CAR) based at least in part on an area of the segmented cribriform morphology and an area of the tumor; and determining a risk of biochemical recurrence (BCR) of a cancer associated with the tumor based at least in part on the CAR.

### CROSS REFERENCE TO RELATED APPLICATIONS

This application claims the benefit of U.S. Provisional Patent Application No. 62/983,939 filed Mar. 2, 2020, entitled “COMPUTERIZED QUANTIFICATION OF INVASIVE CRIBRIFORM ADENOCARCINOMA ON RADICAL PROSTATECTOMY SPECIMENS IS PROGNOSTIC OF BIOCHEMICAL RECURRENCE”, the contents of which are herein incorporated by reference in their entirety.

### FEDERAL FUNDING NOTICE

This invention was made with government support under grants CA202752, CA208236, CA216579, CA220581, and CA239055 awarded by the National Institutes Of Health; grant CON501692 awarded by the National Science Foundation Graduate Research Fellowship Program; grant 1 R43EB028736-01 awarded by the National Institute for Biomedical Imaging and Bioengineering; grant 1 C06 RR12463-01 awarded by the National Center for Research Resources; grant IBX004121A awarded by the United States Department of Veterans Affairs; and grants W81XWH-19-1-0668; W81XWH-15-1-0558, W81XWH-18-1, W81XWH-18-1-0440; and W81XWH-16-1-0329 awarded by the Department of Defense. The government has certain rights in the invention.

### BACKGROUND

There is some evidence that the presence of invasive cribriform adenocarcinoma (ICC) on post-radical prostatectomy (RP) surgical specimens may be correlated with an increased risk of biochemical recurrence (BCR) and metastasis. However, there has been little work on the risk conferred by increasing amount of ICC. Additionally, manual identification of cribriform on H&E (hematoxylin and eosin) stained slides is laborious.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates a flow diagram of an example method/set of operations that can be performed by one or more processors to determine a risk of biochemical recurrence (BCR) of cancer based at least in part on an automatically determined cribriform-to-tumor area ratio, in connection with various aspects discussed herein.

FIG. 2 illustrates a flow diagram of an example method/set of operations that can be performed by one or more processors to train a deep learning model to automatically segment cribriform morphology and/or construct a model that facilitates determining a risk of biochemical recurrence (BCR) of cancer based at least in part on an automatically determined cribriform-to-tumor area ratio, in connection with various aspects discussed herein.

FIG. 3 illustrates example images showing the workflow of the first example use case, in connection with various aspects discussed herein.

FIG. 4 illustrates example images from the first example use case, showing invasive cribriform carcinoma (ICC) regions (top row) and non-ICC regions (middle and bottom rows), in connection with various aspects discussed herein.

FIG. 5 illustrates example images showing the results of ICC segmentation for two patients, in connection with various aspects discussed herein.

FIG. 6 illustrates graphs of Kaplan-Meier survival curves showing the ability of various CAR values to distinguish between BCR and non-BCR patients, in connection with various aspects discussed herein.

FIG. 7 illustrates a table showing results of multivariable cox regression analysis for the first example use case, in connection with various aspects discussed herein.

FIG. 8 illustrates a graph of Kaplan-Meier survival curves for BCR in patients separated based on whether or not the CAR is above 0.0392, in connection with various aspects discussed herein.

FIG. 9 illustrates a graph of Kaplan-Meier survival curves for BCR in patients separated based on different CAR values, in connection with various aspects discussed herein.

FIG. 10 illustrates a table showing a multivariate Cox regression model, in connection with various aspects discussed herein.

FIG. 11 illustrates a chart showing the experimental procedure and the use of patients in the fourth example use case, in connection with various aspects discussed herein.

FIG. 12 illustrates a table summarizing the patient population breakdown using clinical factors, in connection with various aspects discussed herein.

FIG. 13 illustrates a table showing results of univariable and multi-variable analysis for the fourth example use case, in connection with various aspects discussed herein.

FIG. 14 illustrates cribriform segmentation outputs from the D1.1 validation set, in connection with various aspects discussed herein.

FIG. 15 illustrates a diagram of an example apparatus that can facilitate constructing and/or employing a model to predict a likelihood of a biochemical recurrence (BCR) of a tumor based at least in part on a cribriform-to-tumor area ratio (CAR) determined via cribriform morphology automatically segmented via a trained deep learning model, according to various embodiments discussed herein.

### DETAILED DESCRIPTION

Various embodiments discussed herein can construct and/or employ Deep Learning (DL) model(s) to automatically segment cribriform morphology in connection with a tumor and/or determine a likelihood or risk of biochemical recurrence (BCR) of cancer based on a cribriform-to-tumor area ratio (CAR).

Some portions of the detailed descriptions that follow are presented in terms of algorithms and symbolic representations of operations on data bits within a memory. These algorithmic descriptions and representations are used by those skilled in the art to convey the substance of their work to others. An algorithm, here and generally, is conceived to be a sequence of operations that produce a result. The operations may include physical manipulations of physical quantities. Usually, though not necessarily, the physical quantities take the form of electrical or magnetic signals capable of being stored, transferred, combined, compared, and otherwise manipulated in a logic or circuit, and so on. The physical manipulations create a concrete, tangible, useful, real-world result.

It has proven convenient at times, principally for reasons of common usage, to refer to these signals as bits, values, elements, symbols, characters, terms, numbers, and so on. It should be borne in mind, however, that these and similar terms are to be associated with the appropriate physical quantities and are merely convenient labels applied to these quantities. Unless specifically stated otherwise, it is appreciated that throughout the description, terms including processing, computing, calculating, determining, and so on, refer to actions and processes of a computer system, logic, circuit, processor, or similar electronic device that manipulates and transforms data represented as physical (electronic) quantities.

Example methods and operations may be better appreciated with reference to flow diagrams. While for purposes of simplicity of explanation, the illustrated methodologies are shown and described as a series of blocks, it is to be appreciated that the methodologies are not limited by the order of the blocks, as some blocks can occur in different orders and/or concurrently with other blocks from that shown and described. Moreover, less than all the illustrated blocks may be required to implement an example methodology. Blocks may be combined or separated into multiple components. Furthermore, additional and/or alternative methodologies can employ additional, not illustrated blocks.

Embodiments include apparatus, systems, operations, methods, or other embodiments that can construct or employ a deep learning (e.g., a Convolutional Neural Network (CNN) such as a modified UNet, etc.) model to automatically segment cribriform morphology in connection with a tumor and/or determine a likelihood or risk of biochemical recurrence (BCR) of cancer (e.g., prostate cancer, although ICC can potentially be present in other cancer types, e.g., breast cancer, etc.) based on a cribriform-to-tumor area ratio (CAR).

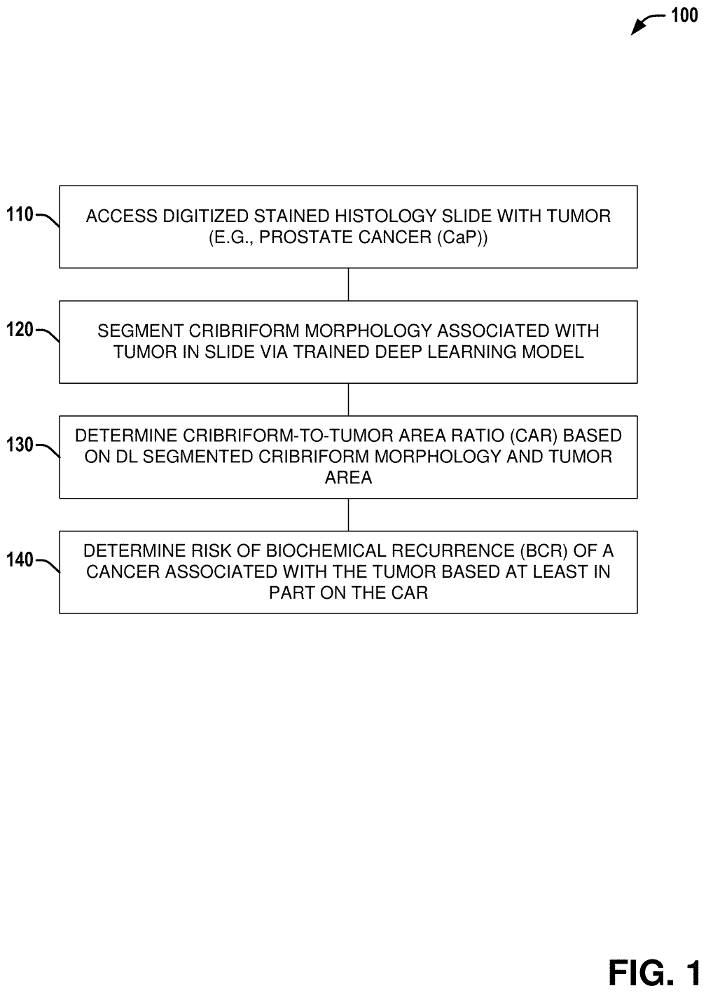

Referring to FIG. 1, illustrated is a flow diagram of an example method/set of operations 100 that can be performed by one or more processors to determine a risk of biochemical recurrence (BCR) of cancer based at least in part on an automatically determined cribriform-to-tumor area ratio, in connection with various aspects discussed herein. Processor(s) can include any combination of general-purpose processors and dedicated processors (e.g., graphics processors, application processors, etc.). The one or more processors can be coupled with and/or can include memory or storage and can be configured to execute instructions stored in the memory or storage to enable various apparatus, applications, or operating systems to perform the operations. The memory or storage devices may include main memory, disk storage, or any suitable combination thereof. The memory or storage devices can comprise—but is not limited to—any type of volatile or non-volatile memory such as dynamic random access memory (DRAM), static random-access memory (SRAM), erasable programmable read-only memory (EPROM), electrically erasable programmable read-only memory (EEPROM), Flash memory, or solid-state storage.

The set of operations 100 can comprise, at 110, accessing at least a portion of a digitized stained histology slide comprising a tumor. In various embodiments and in the example use case discussed below, the digitized stained histology slide can be obtained via a system and/or apparatus implementing the set of operations 100, or can be obtained from a separate medical imaging system (e.g., optical microscope, etc.). Additionally, the digitized stained histology slide can be accessed contemporaneously with or at any point prior to performing the set of operations 100.

The set of operations 100 can further comprise, at 120, automatically segmenting, via a trained deep learning (DL) model, cribriform morphology in connection with the tumor on the at least the portion of the digitized stained histology slide.

The set of operations 100 can further comprise, at 130, determining a cribriform-to-tumor area ratio (CAR) based at least in part on an area of the segmented cribriform morphology and an area of the tumor.

The set of operations 100 can further comprise, at 140, determining a risk of biochemical recurrence (BCR) of a cancer associated with the tumor based at least in part on the CAR.

Additionally, or alternatively, set of operations 100 can comprise one or more other actions discussed herein in connection with determining a prognosis and/or risk of BCR in connection with segmented cribriform morphology of tumor histology.

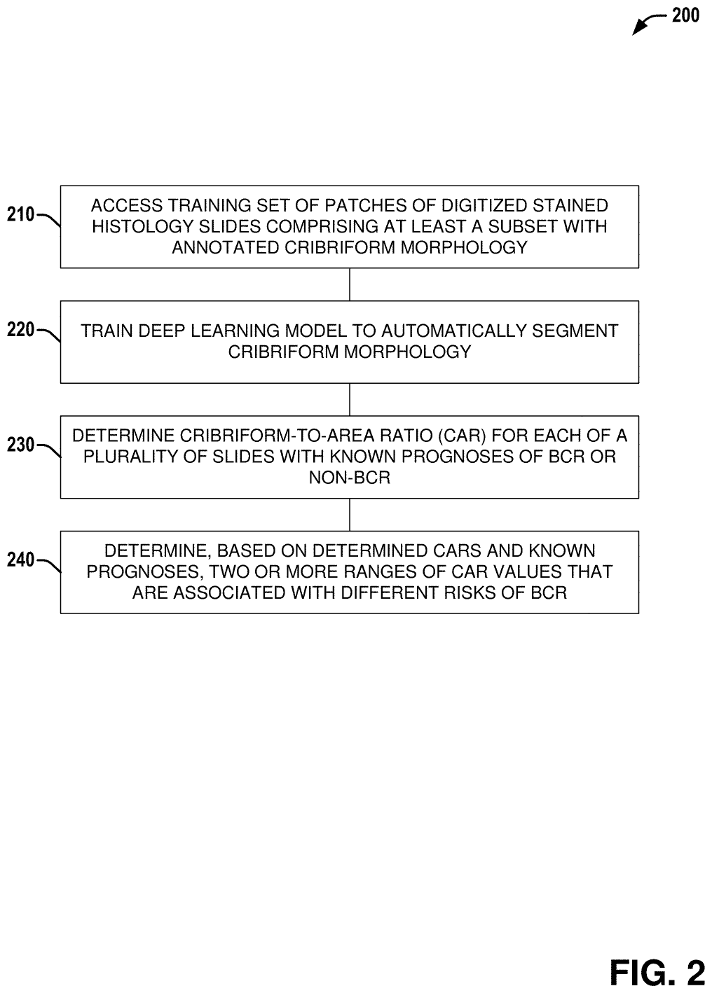

Referring to FIG. 2, illustrated is a flow diagram of an example method/set of operations 200 that can be performed by one or more processors to train a deep learning model to automatically segment cribriform morphology and/or construct a model that facilitates determining a risk of biochemical recurrence (BCR) of cancer based at least in part on an automatically determined cribriform-to-tumor area ratio, in connection with various aspects discussed herein.

The set of operations 200 can comprise, at 210, accessing a training set of patches, wherein each patch is extracted from an associated digitized stained histology slide of a first plurality of digitized stained histology slides, wherein each digitized stained histology slide of the first plurality of digitized stained histology slides comprises an associated tumor, and wherein each patch of a first subset of the training set comprises cribriform morphology that has been annotated on that patch. In various embodiments, the digitized slides can be obtained via a system and/or apparatus implementing the set of operations 200, or can be obtained from a separate medical imaging system (e.g., optical microscopy system). Additionally, the digitized slides can be accessed contemporaneously with or at any point prior to performing the set of operations 200.

The set of operations 200 can further comprise, at 220, training a deep learning (DL) model to automatically segment cribriform morphology based at least in part on the training set of patches and the annotated cribriform morphology.

The set of operations 200 can further comprise, at 230, determining via the trained DL model, for each digitized stained histology slide of a second plurality of digitized stained histology slides, an associated cribriform-to-tumor area ratio (CAR) for that digitized stained histology slide, wherein each digitized stained histology slide of the second plurality of digitized stained histology slides is associated with a known prognosis of biochemical recurrence (BCR) or non-BCR. In various aspects, the second plurality can be the first plurality, can be a subset of the first plurality, can comprise one or more slides of the first plurality and one or more other slides, or can have no slides in common with the first plurality.

The set of operations 200 can further comprise, at 240, determining two or more ranges of CAR values based on the associated CARs and known prognoses for the second plurality, wherein each range of CAR values of the two or more ranges of CAR values is associated with a different risk of BCR.

Additionally, or alternatively, set of operations 200 can comprise one or more other actions discussed herein in connection with training a deep learning model to automatically segment cribriform morphology and/or constructing a model that facilitates determining a risk of biochemical recurrence (BCR) of cancer based at least in part on an automatically determined cribriform-to-tumor area ratio.

Additional aspects and embodiments are discussed below in connection with the following example use cases.

### EXAMPLE USE CASES

### Computerized Quantification of Invasive Cribriform Adenocarcinoma on Radical Prostatectomy Specimens is Prognostic of Biochemical Recurrence

### Example Use Case 1

The following discussion provides example embodiments in connection with a first example use case involving using deep learning to automatically segment cribriform morphology and using the cribriform-to-tumor area ratio (CAR) to predict biochemical recurrence (BCR) In prostate cancer (CaP).

### Introduction

Objective: To automatically quantify invasive cribriform carcinoma (ICC) on diagnostic H&E radical prostatectomy (RP) slides and assess its prognostic ability of biochemical recurrence (BCR).

Overview: ICC in RP specimens has been correlated with BCR and metastasis of prostate cancer (CaP). Manual quantification of cribriform on H&E images is laborious.

Approach: Train a deep learning (DL) model to segment regions of ICC, determine the cribriform-to-tumor area ratio (CAR), and assess if CAR is prognostic of BCR.

### Methodology

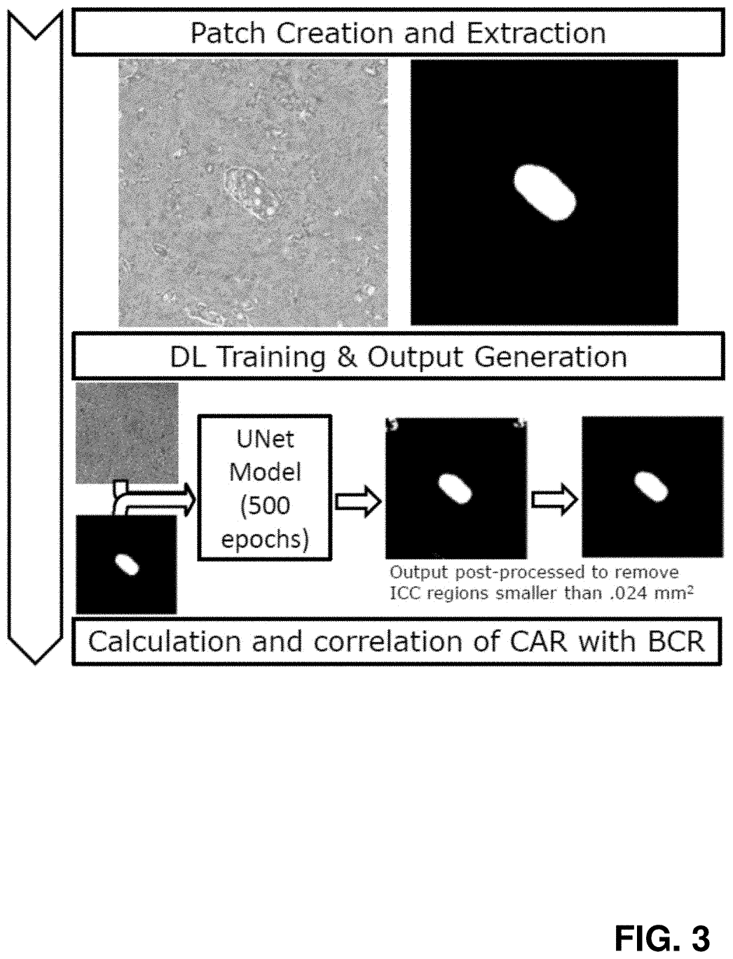

Referring to FIG. 3, illustrated are example images showing the workflow of the first example use case, in connection with various aspects discussed herein. In the top row, images show patch creation and extraction (e.g., with an example image (left) and example ICC expert annotation and/or segmentation (right)). The bottom row shows images representing DL training and output generation. Training images and associated annotation/segmentation can be provided to a DL model to be trained on it (e.g., the UNet model of the first example use case, which was trained for 500 epochs, and the model associated with the lowest test set loss was selected. In other embodiments, other DL models can be employed.). The trained DL model can generate output images indicating ICC regions, as seen in the second image from the right on the bottom row. Additional post-processing can be employed to remove ICC regions smaller than a threshold (e.g., 0.024 mm², as in the first example use case, or larger or smaller (e.g., 0.01, 0.02, 0.03, 0.04, etc.), to obtain a post-processed image (e.g., the example image at bottom right). After output generation, ICC regions and tumor area of images of the test set can be used to generate a CAR for that image, and correlation with BCR can be determined.

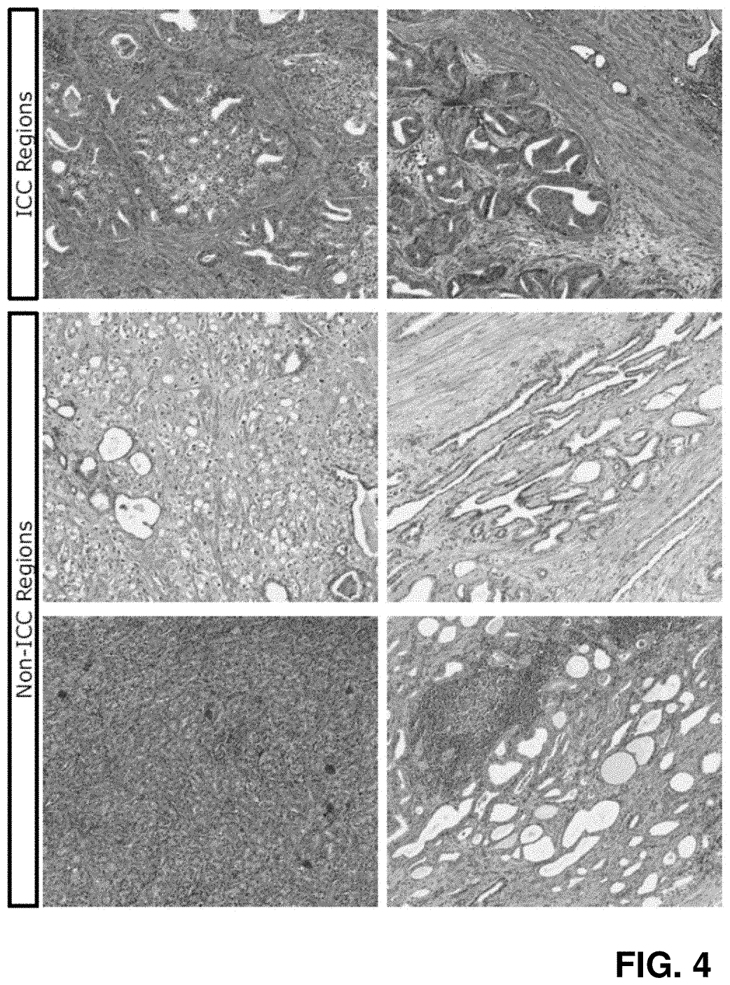

The first example use case used imaging from N=703 RP patients from six institutions, 69 for training, 634 for testing. 346 1 k by 1 k pixel patches at 10× magnification were extracted containing ICC and non-ICC regions. Referring to FIG. 4, illustrated are example images from the first example use case, showing invasive cribriform carcinoma (ICC) regions (top row) and non-ICC regions (middle and bottom rows), in connection with various aspects discussed herein. The top row of FIG. 4 shows samples of cribriform patches identified by expert pathologists in the training set (N=69). The middle and bottom rows of FIG. 4 show sample patches of non-ICC lesions to increase morphological diversity for model diversity.

In the first example use case, DL training was performed on N=33 patients, and DL testing was performed on N=3 patients. CAR was calculated on N=703 patients by dividing the area of ICC by tumor area. A CAR threshold was identified based on the training set, and applied on the testing set for prediction of BCR. Table 1, below, shows the dataset employed in the first example use case:

TABLE 1
First Example Use Case Dataset Description

| | n (n BCR) | Censor (BCR) year |
|---|---|---|
| Train | 69 (35) | 1.67 (1.73) |
| Test | 634 (219) | 2.25 (1.75) |

### Results

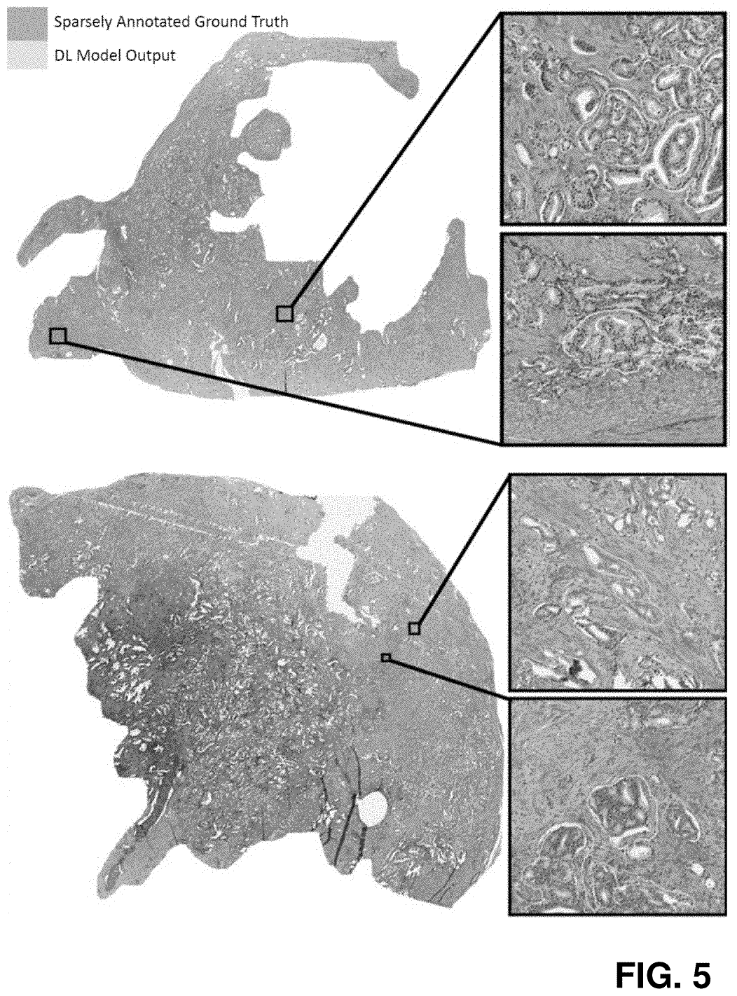

The ICC segmentation model achieved a per pixel sensitivity and specificity of 95% and 80% respectively. Referring to FIG. 5, illustrated are example images showing the results of ICC segmentation for two patients, in connection with various aspects discussed herein. In FIG. 5, green boundaries correspond to sparse pathologist annotations, while yellow boundaries correspond to DL-predicted ICC regions.

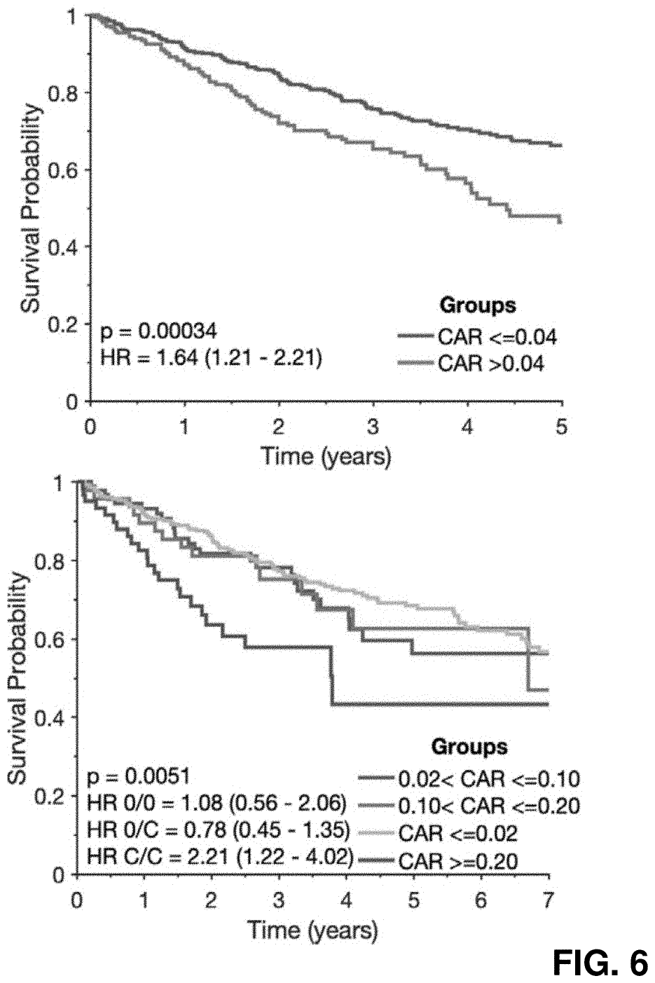

For the first example use case, a CAR threshold of 0.04 was best shown to separate out BCR and non-BCR patients (various embodiments can employ other thresholds, such as those discussed herein (e.g., 0.0392) or greater or lesser values, etc.). Referring to FIG. 6, illustrated are graphs of Kaplan-Meier survival curves showing the ability of various CAR values to distinguish between BCR and non-BCR patients, in connection with various aspects discussed herein. The top chart shows BCR survival curves of patients using CAR with a threshold of 0.04. The bottom chart shows Kaplan-Meier BCR survival curves of patients using CAR ≤0.02, CAR 0.02-0.1, CAR 0.1-0.2, and CAR ≥0.2 on the test set (N=634). As seen in FIG. 6, CAR (p-value=0.002, HR=1.61, 95% CI: 1.18-2.19) was independently prognostic of BCR in the test set (N=634).

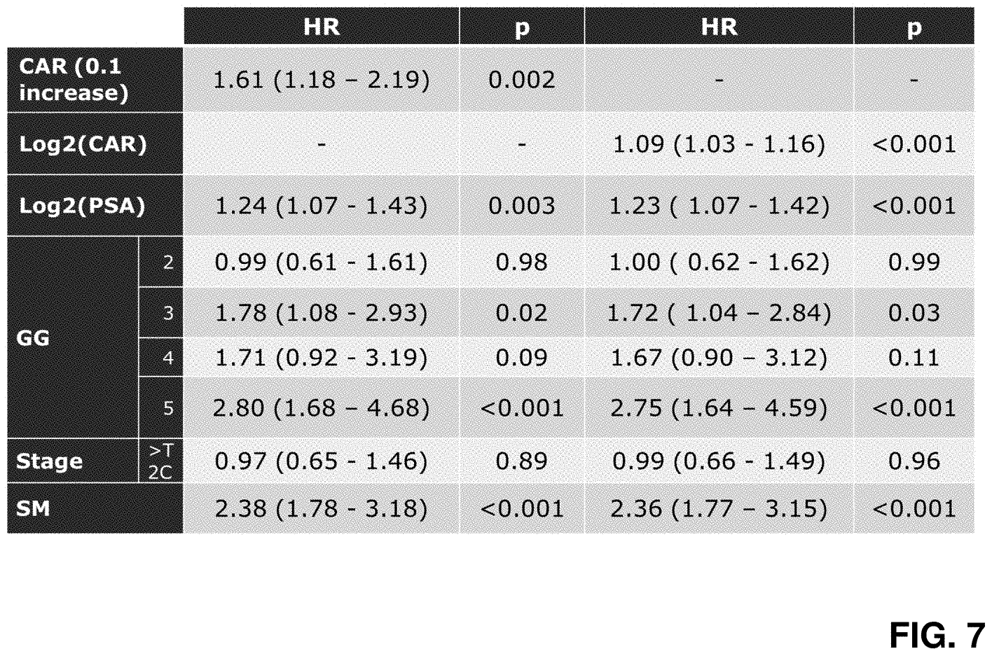

Referring to FIG. 7, illustrated is a table showing results of multivariable cox regression analysis for the first example use case, in connection with various aspects discussed herein. CAR correlated with BCR when accounting for unclean surgical margins and International Society of Urological Pathologists (ISUP) Grade Group (GG) (also referred to herein as Gleason GG) 5.

### Conclusion

CAR from H&E post-RP slides is prognostic of BCR. The first example use case was limited in that immunohistochemistry (IHC) was not performed to differentiate and study both ICC and intraductal carcinoma of the prostate (IDC-P), and that metastasis information was unavailable for the group of patients of the first example use case.

### Example Use Case 2

The following discussion provides example embodiments in connection with a second example use case involving using deep learning to automatically segment cribriform morphology and using the cribriform-to-tumor area ratio (CAR) to predict biochemical recurrence (BCR) In prostate cancer (CaP).

### Introduction

Invasive cribriform adenocarcinoma (ICC) in radical prostatectomy (RP) specimens has been correlated with biochemical recurrence (BCR) and metastasis of prostate cancer (CaP). However, manual identification of cribriform on H&E slides is laborious. To quantify extent of ICC across many patients, an automated method is needed. The second example use case trained a deep learning (DL) model to segment ICC on diagnostic H&E-stained RP slides and assessed cribriform-to-tumor area ratio (CAR) as a predictor of BCR.

### Methods:

A single H&E-stained diagnostic slide was collected from each of N=261 RP patients from a single institution. All slides were annotated for a representative tumor region. The training dataset, D1, was composed of N=69 patients. The remaining N=192 patients composed the validation set D2. Two pathologists reviewed D1 and sparsely annotated ICC regions on 30 patients using standard criteria for cribriform carcinoma. A 1000×1000-pixel region at 10× magnification was extracted from 325 ICC lesions. To increase diversity in the negative class of non-ICC tissue presentation, 21 1000×1000 pixel regions were manually selected from 6 additional patients. These patches included common confounding cribriform patterns such as Grade 5 (excluding Comedonecrosis) and pseudo-cribriform areas along with lymphocytic regions and benign glands. The 36 patients were randomly divided into training (33 patients) and testing (3 patients) sets. Training set patches were augmented prior to use in a UNet DL model. After training for 500 epochs, the model associated with the lowest test set loss was selected. This model was then applied to the representative tumor region on each slide. Model output was post-processed to remove areas smaller than the smallest ICC area in the training set, 0.024 mm². CAR was calculated for every patient. D1 was used to identify a threshold of CAR best separating outcomes and then subsequently applied to patients in D2.

### Results:

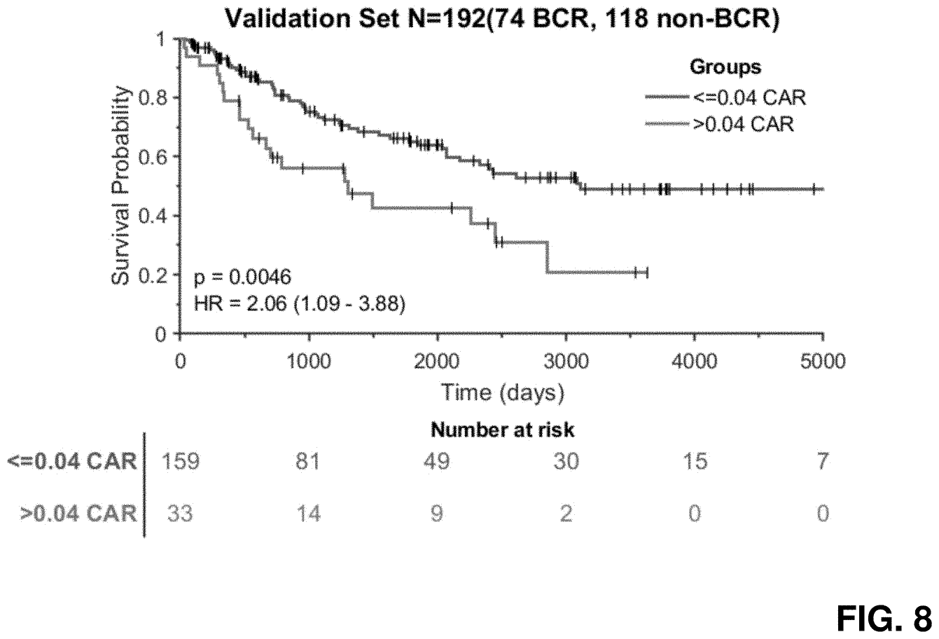

The ICC segmentation model achieved a per pixel sensitivity and specificity of 95% and 80%, respectively, in the test set (see FIG. 5, discussed above). A CAR threshold of 0.039 was best shown to separate out BCR and non-BCR patients. CAR (p-value<0.0046, HR=2.06, 95% CI: 1.09-3.88) was prognostic of BCR in D2. Referring to FIG. 8, illustrated is a graph of Kaplan-Meier survival curves for BCR in patients separated based on whether or not the CAR is above 0.0392, in connection with various aspects discussed herein.

### Conclusion:

The second example use case demonstrated that an automatically derived CAR from H&E post-RP slides is prognostic of BCR. Limitations to the second example use case include that IHC was not performed to differentiate and study both ICC and intraductal carcinoma of the prostate (IDC-P). Also, the second example use case did not incorporate a larger multiinstitutional validation set and IHC.

### Example Use Case 3

The following discussion provides example embodiments in connection with a third example use case involving using deep learning to automatically segment cribriform morphology and using the cribriform-to-tumor area ratio (CAR) to predict biochemical recurrence (BCR) In prostate cancer (CaP).

### Introduction

There is some evidence that the presence of invasive cribriform adenocarcinoma (ICC) on post-radical prostatectomy (RP) surgical specimens may be correlated with an increased risk of biochemical recurrence (BCR) and metastasis. However, there has been little work on the risk conferred by increasing amount of ICC. The third example use case developed a deep learning (DL) model to segment ICC on diagnostic H&E RP slides and assessed the correlation between cribriform-to-tumor area ratio (CAR) and BCR.

### Methods:

A single H&E slide was gathered from each of N=933 patients from 6 institutions. Each slide was annotated for a single representative tumor region. Patients from one institution comprised the training cohort D1 (N=69) with the remainder in the testing cohort (D2). From 30 patients of D1, 325 1000×1000 pixel regions at 10× magnification were extracted and annotated for ICC. 21 regions were also selected from another 6 patients to increase diversity in the negative class. These included non-ICC Gleason 5 patterns (excluding comedonecrosis), pseudo-cribriform, lymphocytic regions, and benign glands. These 376 1000×1000 pixel regions from 36 patients were used to train a UNet model for ICC segmentation. The set of weights yielding the lowest test loss was selected as the final model. This model was applied to the representative tumor region on all N=933 patients. Model output was post-processed to remove areas smaller than the smallest ICC area in D1.1 (0.024mm²). CAR was then calculated for every patient as the detected ICC area divided by the total annotated tumor area. Three CAR thresholds were empirically chosen from D1 and applied to D2 to study its effects on BCR risk. A multivariable cox regression model was performed to study CAR in the presence of other confounding variables.

### Results:

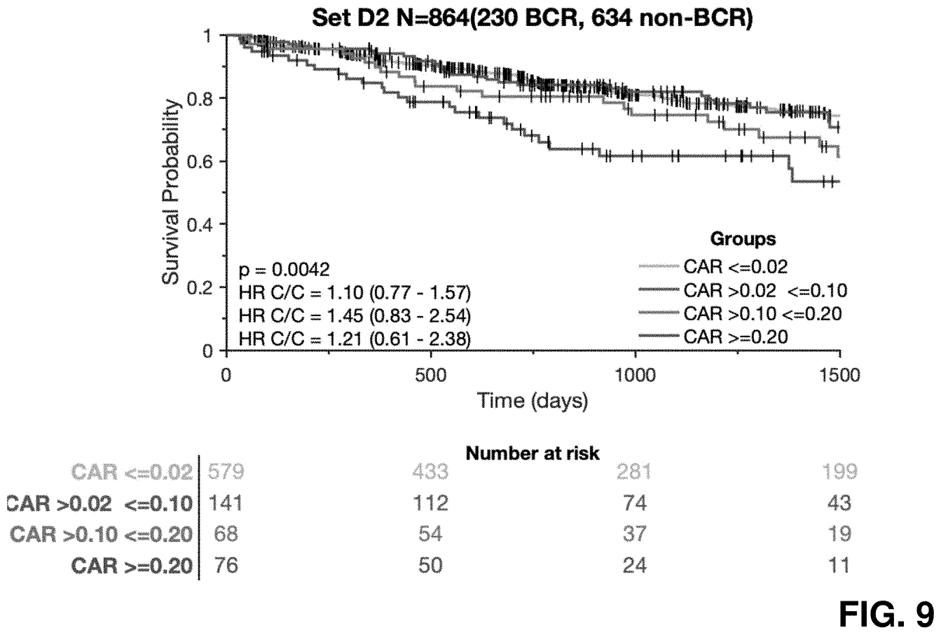

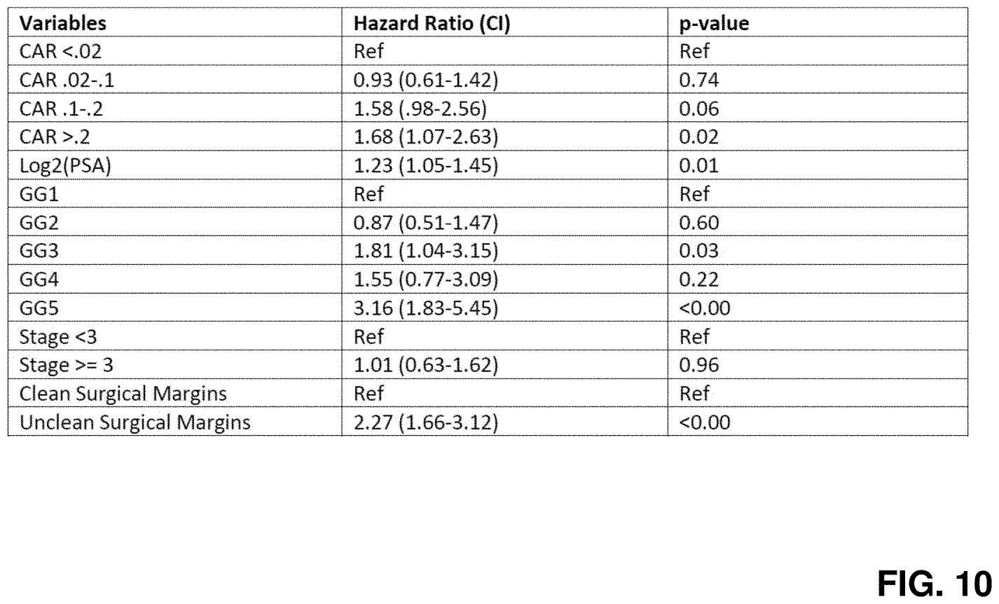

The UNet model achieved a per pixel sensitivity and specificity of 95% and 80% respectively on D1.1 (see FIG. 5). The three thresholds for CAR were significant (p-value<0.0042) for BCR prognosis in D2. Referring to FIG. 9, illustrated is a graph of Kaplan-Meier survival curves for BCR in patients separated based on different CAR values, in connection with various aspects discussed herein. FIG. 9 shows Kaplan-Meier BCR survival curves of patients using CAR <0.02, CAR 0.02-0.1. CAR 0.1-0.2, and CAR >0.2 on validation set D2. The curves are truncated at 1500 days. When controlling for other confounder variables like surgical margin and Gleason grade group 5, an increase in CAR was correlated with an increase in hazard ratio. Referring to FIG. 10, illustrated is a table showing a multivariate Cox regression model, in connection with various aspects discussed herein. An increase in CAR was correlated with an increase in hazard ratio (HR) when accounting for unclean surgical margins and Gleason Grade Group (GG) 5.

### Conclusion:

The third example use case demonstrated that an increase in CAR is associated with an increased risk of BCR post-RP. Limitations to the third example use case include that IHC was not performed to differentiate ICC and intraductal carcinoma of the prostate (IDC-P).

### Example Use Case 4

The following discussion provides example embodiments in connection with a fourth example use case involving using deep learning to automatically segment cribriform morphology and using the cribriform-to-tumor area ratio (CAR) to predict biochemical recurrence (BCR) In prostate cancer (CaP).

### 1. Introduction:

Clinicians decide treatment options for patients with prostate cancer (CaP) with several clinical factors including tumor stage, prostate specific antigen (PSA), and an evaluation of tissue architecture. Tissue architecture is described numerically on a scale from 1 to 5 by identifying the two most recurring architectural patterns found on a tissue specimen, the sum of which is stratified into different Gleason grade groups [2]. These patterns are each stratified into a score ranging from 1 to 5, the sum of which is categorized into different Gleason grade groups. Grade group 1 patients have good clinical outcomes and grade group 4-5 patients have poor clinical end points, however the spectrum in between have variable outcomes. Cribriform, currently classified as a Gleason 4 pattern, has been correlated with increasingly adverse outcomes comparable to those observed in grade group 5.

Cribriform architecture is characterized by an expansile proliferation of carcinoma cells the size of at least a benign gland containing multiple lumina and no intervening stroma. Its incidence rate can be as low as 2.1% in low-risk cohorts or as high as 56% in high-risk cohorts. Patients post-RP may experience a relapse in disease signaled by an increase in PSA called biochemical recurrence (BCR) and would then subsequently require adjuvant therapy. Physicians are therefore forced to choose between the overtreatment of low-risk BCR patients and the risk of possible clinical progression associated with delaying adjuvant therapy. An early risk assessment for the onset of BCR post-RP is vital for physicians to create a comprehensive treatment plan. Several studies have shown that the presence of cribriform morphology post-radical prostatectomy (RP) has been strongly correlated with biochemical recurrence (BCR) and metastasis. While cribriform morphology is significantly correlated with poor clinical endpoints, there exists strong difficulty in distinguishing clinical implications solely based on presence of cribriform morphology. Specific cribriform patterns lie on a varying spectrum ranging from benign to malignant each having different implications on risk of BCR and metastasis. Differentiating between specific cribriform patterns on an H&E slide is therefore challenging and sometimes impossible without immunohistochemistry (IHC).

Deep learning (DL) is used in the field of digital pathology to identify common primitives found on H&E slides such as nuclei and lumen. DL models utilize a vast amount of data to implicitly learn important features capable of accurately identifying a specific pattern. DL models have been used in several contexts including nuclear and luminal segmentation, tissue classification, and IHC staining.

The fourth example use case trained a deep learning model capable to identify and segment invasive cribriform carcinoma patterns. Using these segmentation results, a threshold of cribriform area was identified that best differentiates BCR and non-BCR patients. These results were validated on a large multi-institutional dataset. Finally, the fourth example use case demonstrates that the amount of invasive cribriform carcinoma can go beyond traditionally used risk-stratification tools in discriminating BCR vs non-BCR patients (e.g., determining a BCR risk category based on a CAR value falling into one of a plurality of bins or ranges, etc.).

### 2. Materials and Methods

### 2.1 Patient Cohorts

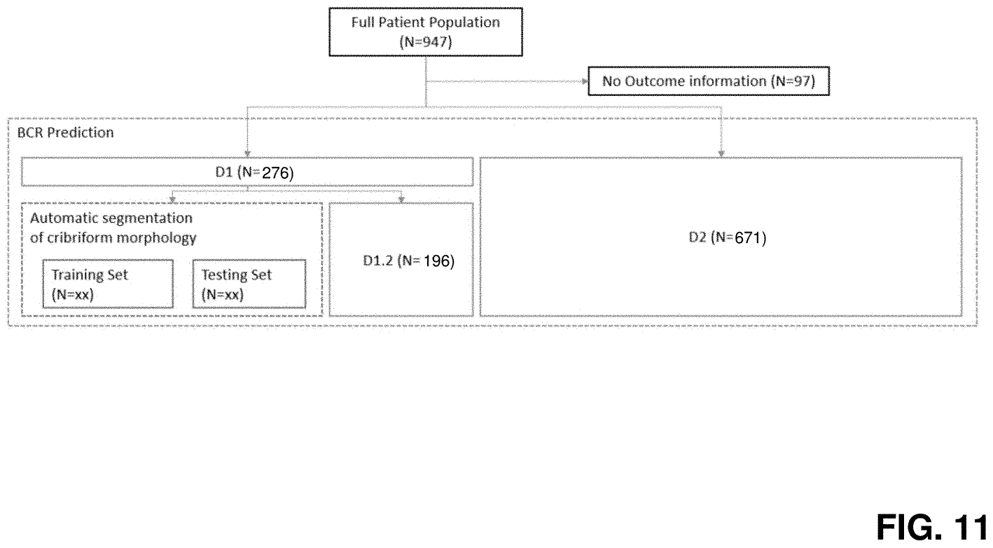

This study collected N=947 patients from five institutions: University of Pennsylvania (UPenn), Cornell University, University of Turku (UTurku), the Cancer Genomic Atlas (TCGA), and University Hospitals (UH). These patients were split into a training (D1) and a hold-out validation (D2) cohort. The N=276 patients from UPenn composed D1 and the remainder N=671 patients composed D2. A subset of D1 (N=69) patients with BCR information were used to train the ICC segmentation model. However, only 30 of these patients expressed ICC morphology. To increase the diversity of tissue architecture for the segmentation model, 6 additional patients from D1 were included. The entirety of cohort D1 was used to train a BCR prediction model. This model was validated on cohort D2. Referring to FIG. 11, illustrated is a chart showing the experimental procedure and the use of patients in the fourth example use case, in connection with various aspects discussed herein.

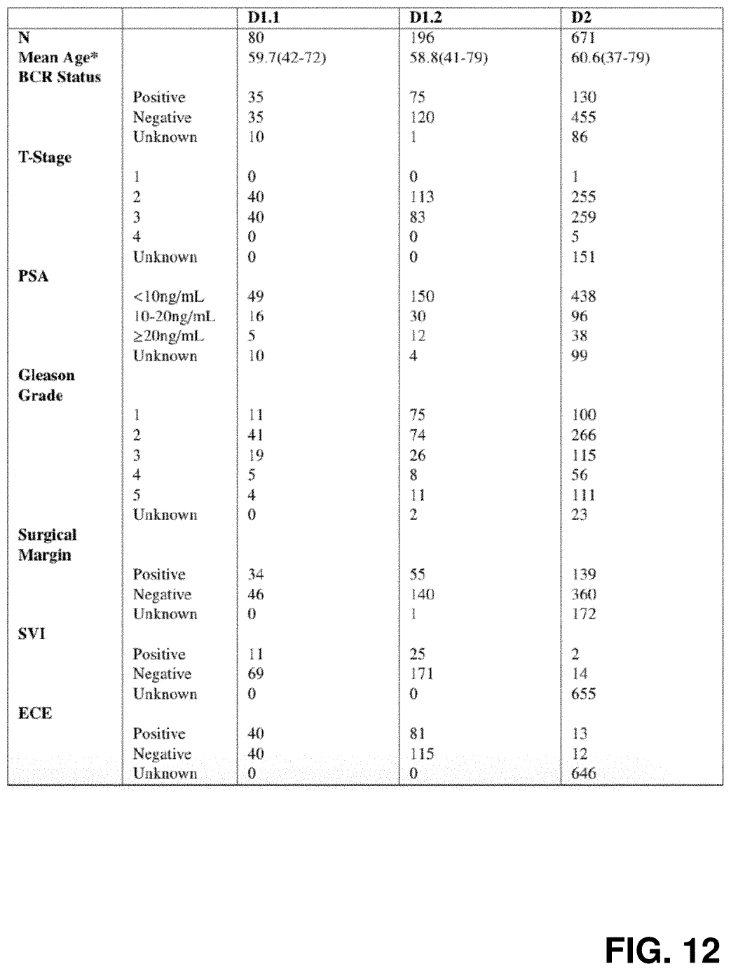

Patients were labeled as BCR if they expressed two consecutive PSA tests results >0.2 ng/mL and their time to BCR was recorded on the second PSA failure date. Non-BCR patients were censored on the last available PSA test date. None of the patients in this study received neo-adjuvant or adjuvant therapy before BCR failure or censored date. Referring to FIG. 12, illustrated is a table summarizing the patient population breakdown using clinical factors, in connection with various aspects discussed herein. Not all patients had age information to calculate an average and range. Cohort D1 was comprised of subsets D1.1 and D1.2. Manual analysis of cribriform morphology on whole mount H&E slides were conducted only on subset D1.1. Of these 80 patients, 23 patients presented cribriform morphology.

As noted, manual detection of cribriform morphology was performed only on the subset D1.1 (N=80). A first pathologist analyzed all 80 patients and identified 30 patients expressing adenocarcinoma cribriform morphology as defined by standard cribriform carcinoma selection criteria. The first pathologist non-exhaustively annotated these 30 patients. A 1000×1000 pixel patch at 10× (1 MPP) centered around each area of cribriform was extracted and a second pathologist exhaustively annotated each of these patches for all areas of cribriform morphology using the same criteria. A total of 326 patches were used to train the cribriform segmentation model.

### 2.2 Automatic Segmentation of Cribriform Morphology

Cribriform glands were segmented using a UNet-inspired DL architecture. Only cohort D1.1 was used to train and validate the segmentation model. The model was trained using 512 by 512 pixel patches extracted randomly from 1000 by 1000 pixel ROIs at 10× magnification (1 MPP) from 33 patients. The model was validated on 3 patients. No patients were shared between training and validation. Several spatial and color augmentations were performed using the albumentations library. The 100 k parameter model was trained using an Adam loss function for 500 epochs. The model that yielded the lowest validation loss was regarded as the final model used throughout the fourth example use case. The model was then subsequently run on all 807 patients to generate a binary mask indicating regions of cribriform. Regions with an area smaller than the smallest cribriform area in the dataset, 0.024 mm², were removed. Binary mask outputs on D1.1 were qualitatively verified by the second pathologist to be accurate. A per pixel sensitivity and specificity were calculated on the validation set to quantitatively compare model output to the ground truth.

### 2.3 BCR Prognosis

Using the cribriform segmentations for all 807 cases, the percent of cribriform area within the cancerous region with respect to the amount of cancer area was determined. This was the only feature that was considered in the fourth example use case. The threshold of cribriform area that maximizes separability between BCR and non-BCR patients was determined on cohort D1. This threshold was then applied onto cohort D2. Logrank p-value and hazard ratio were used to evaluate the ability of the percent cribriform area to discriminate between BCR and non-BCR patients. Univariable and multivariable cox regression models were used to evaluate CAR independently and against other confounders. Confounders include PSA, GG, tumor stage, and presence of surgical margins. CAR was evaluated continuously and categorically.

### 3. Results

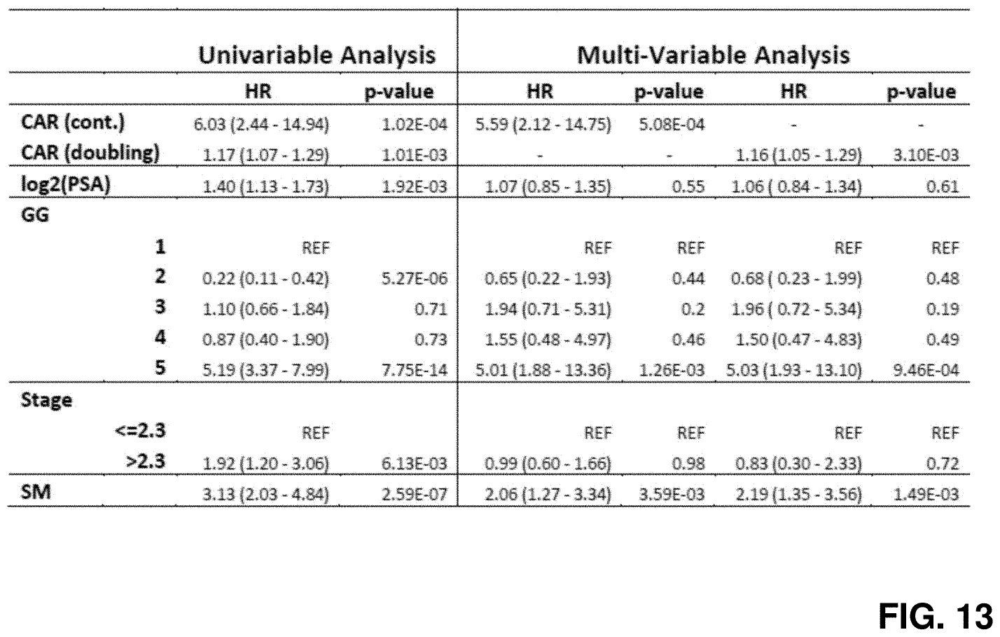

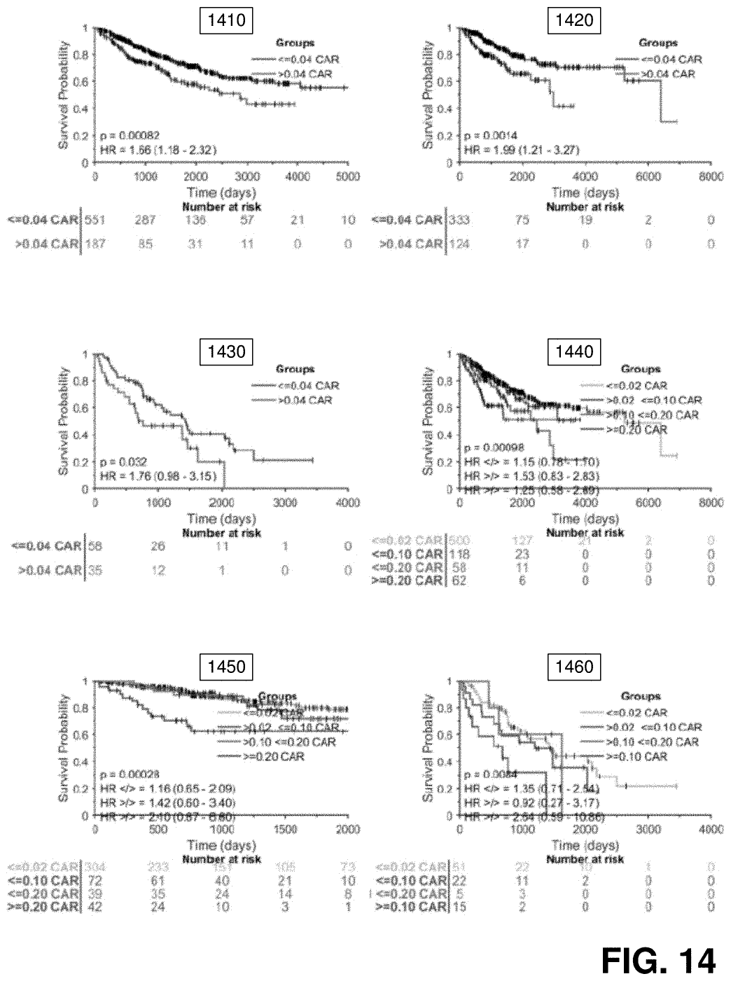

Referring to FIG. 13, illustrated is a table showing results of univariable and multi-variable analysis for the fourth example use case, in connection with various aspects discussed herein. Referring to FIG. 14, illustrated are cribriform segmentation outputs from the D1.1 validation set, in connection with various aspects discussed herein. 1410 shows Kaplan-Meier curves of CAR on the full validation set with a threshold of 0.039. 1420 shows the CAR threshold applied on patients with a clean surgical margin on the validation set. 1430 shows the CAR with a threshold of 0.039 on the Gleason grade group 5 patients in the validation set. 1440 shows multiple CAR thresholds empirically derived from the training sets dot plot and applied to the full validation set. 1450 shows the same multiple thresholds applied to the subset of validation set possessing clean surgical margins. 1460 shows the multiple thresholds applied to the Gleason grade group 5 patients in the validation set.

Within D1, a CAR of 0.0681 was found to best discriminate between patients expressing BCR and patients that did not. 33.87% of patients that recurred within D2 presented cribriform morphology above this threshold, however only 19.49% of patients that did not recur had a CAR value greater than 0.0681. CAR was significant in predicting BCR in D1 and in D2. CAR was also significant in BCR prognosis in intermediate and high grade groups but did not demonstrate an increase in HR. CAR had an increase in HR amongst patients with negative surgical margins within D1 and D2. The survival curves using CAR are shown in FIG. 14 for multiple clinical risk stratifications.

The continuous and the doubling CAR cox regression models were also prognostic independent of PSA, GG, tumor stage, and presence of surgical margins, as shown in FIG. 13. Accounting for other confounders, CAR was significant and prognostic in both the categorical and the doubling models as shown also in FIG. 13.

### 4. Discussion

While RP is the most common primary treatment for select patients with intermediate to high grade disease, as high as 45% of post-RP patients experience BCR. BCR is determined by an increase in PSA levels >0.2 ng/mL, however the outlook of BCR varies considerably, so not all of these patients will experience clinical progression requiring secondary treatment. With a 5-year disease specific survival of 93% for PSA-defined BCR patients, a better risk stratification technique than existing techniques can identify higher-risk individuals. The demerits of assessing BCR using PSA levels is further demonstrated by the possibility that PSA may be produced by benign prostate tissue left over post-RP. Furthermore, the efficacy of adjuvant therapy is maximized within an early period post-RP for high-risk patients making the early identification of this subset important. Identifying accurate and robust BCR risk factors could reduce overtreatment of low-risk patients while directing appropriate adjuvant therapy for high-risk individuals.

Recently, studies have reported that Gleason score is significant in stratifying individuals for the risk of disease specific mortality. However, Gleason score is subject to a high inter-reviewer variability. Therefore, there has been increased effort to go beyond Gleason scoring and directly correlate specific architectural patterns on H&E slides to risk of BCR and overall survival. Cribriform morphology, currently a Gleason 4 pattern, is significantly correlated with BCR and metastasis. One study reported cribriform morphology to be found in 61% of BCR patients but in only 16% of non-BCR cases. Another study found cribriform morphology in 81% of all metastatic cases. Cribriform patterns were prognostic of BCR and metastasis with an odds ratio (OR) of 3.3 and 8.0 respectively, making cribriform an important marker to study.

While cribriform patterns have a diagnostic concordance of 79% among pathologists when compared to other Gleason 4 patterns, the manual identification and segmentation of cribriform morphology on an H&E slide is a time intensive and laborious process. The effects of cribriform morphology have also not been fully studied. While many studies have reported on the correlation between the presence of cribriform and adverse outcomes, very few (if any) studies have studied the effects of the amount of cribriform morphology present. Understanding cribriform can also be difficult, as its morphology can vary from benign to malignant, each type having different diagnostic implications.

In the fourth example use case, a deep learning (DL) model was trained to segment regions of ICC on diagnostic H&E-stained RP slides and subsequently evaluated the CAR in BCR prognosis. The fourth example use case also evaluated the effects of increasing CAR on BCR-risk. Like previously reported studies, the segmentation model identified ICC in 58% of all BCR patients. On only the TCGA dataset, the model identified ICC patterns on 38% of cases, a finding similar to a study where pathologists manually identified ICC patterns on 31% of cases. CAR was prognostic of BCR and was significant when accounting for Gleason grade group, preop-PSA, tumor stage, and SM. CAR was independently more prognostic than Gleason grade 5, a finding consistent with a cribriform study on needle-core biopsies placing the importance of cribriform morphology at the same level as Gleason 5 patterns. CAR was not prognostic (p>0.05) in low Gleason grade groups. This may be because low Gleason grade groups lack substantial ICC patterns and other markers played an increased role in determining risk.

The fourth example use case also analyzed the effects of increasing CAR on BCR prognosis. There was a greater degree of separability between a threshold at 5% versus 20% suggesting that an increased ICC area on a tumor slide is correlated with an increased risk of BCR. This was further confirmed in the cox regression model indicating that an increase in the ICC area by a factor of 2 while accounting for other confounders is correlated with a 17% increase in risk of developing BCR. This finding is similar to a study that found that the OR of failure was 1.173 for every square millimeter of cribriform area added. This suggests that there is significant value for BCR prognosis in quantifying the amount of ICC area present and not just analyzing the presence it.

The fourth example use case also had several limitations that can be addressed in various embodiments. While the dataset was specifically annotated for ICC morphology only, in some cases it was impossible to conclusively differentiate ICC and IDC on an H&E stain alone. Various embodiments can incorporate IHC stains to create a training and testing set that only includes ICC. Metastasis information was unavailable in the dataset and therefore limited feedback was provided on the need for adjuvant therapy. While a qualitative examination on random subsets in the validation set showed that the ICC segmentation model performed reasonably, BCR prognosis was not significant in some subsets within the validation set.

### Additional Embodiments

In various example embodiments, method(s) discussed herein can be implemented as computer executable instructions. Thus, in various embodiments, a computer-readable storage device can store computer executable instructions that, when executed by a machine (e.g., computer, processor), cause the machine to perform methods or operations described or claimed herein including operation(s) described in connection with methods 100, 200, or any other methods or operations described herein. While executable instructions associated with the listed methods are described as being stored on a computer-readable storage device, it is to be appreciated that executable instructions associated with other example methods or operations described or claimed herein can also be stored on a computer-readable storage device. In different embodiments, the example methods or operations described herein can be triggered in different ways. In one embodiment, a method or operation can be triggered manually by a user. In another example, a method or operation can be triggered automatically.

Embodiments discussed herein relate to constructing and/or employing models that can determine a risk of biochemical recurrence (BCR) based at least in part on cribriform morphology segmented via deep learned features or mappings that are not perceivable by the human eye, and involve computation that cannot be practically performed in the human mind. As one example, deep learning models as described herein cannot be implemented in the human mind or with pencil and paper. Embodiments thus perform actions, steps, processes, or other actions that are not practically performed in the human mind, at least because they require a processor or circuitry to access digitized images stored in a computer memory and to extract or compute features that are based on the digitized images and not on properties of tissue or the images that are perceivable by the human eye. Embodiments described herein can use a combined order of specific rules, elements, operations, or components that render information into a specific format that can then be used and applied to create desired results more accurately, more consistently, and with greater reliability than existing approaches, thereby producing the technical effect of improving the performance of the machine, computer, or system with which embodiments are implemented.

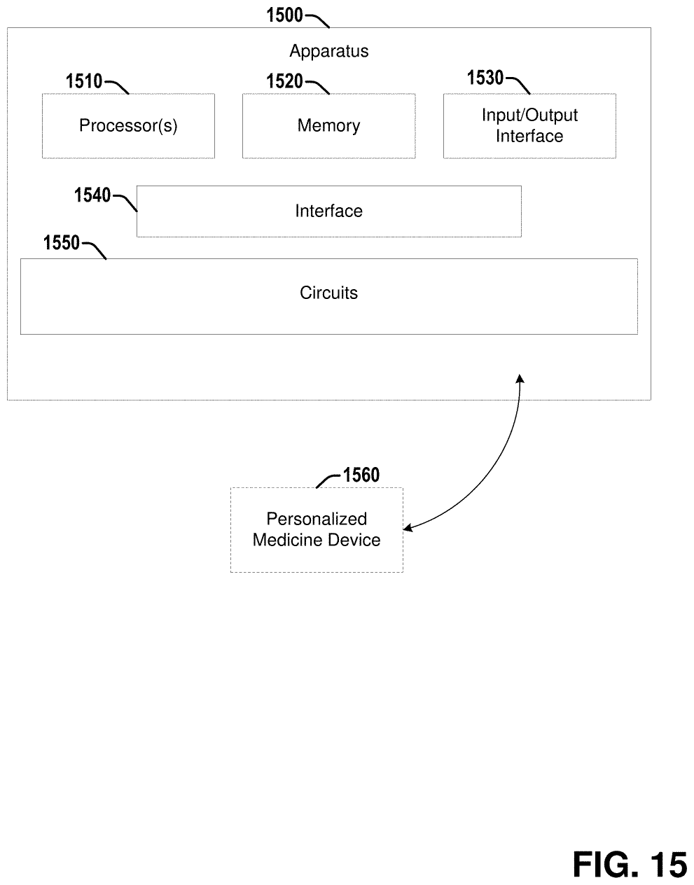

Referring to FIG. 15, illustrated is a diagram of an example apparatus 1500 that can facilitate constructing and/or employing a model to predict a likelihood of a biochemical recurrence (BCR) of a tumor based at least in part on a cribriform-to-tumor area ratio (CAR) determined via cribriform morphology automatically segmented via a trained deep learning model, according to various embodiments discussed herein. Apparatus 1500 can be configured to perform various techniques discussed herein, for example, various operations discussed in connection with sets of operations 100, 200, and/or other methods described herein. Apparatus 1500 can comprise one or more processors 1510 and memory 1520. Processor(s) 1510 can, in various embodiments, comprise circuitry such as, but not limited to, one or more single-core or multi-core processors. Processor(s) 1510 can include any combination of general-purpose processors and dedicated processors (e.g., graphics processors, application processors, etc.). The processor(s) can be coupled with and/or can comprise memory (e.g., of memory 1520) or storage and can be configured to execute instructions stored in the memory 1520 or storage to enable various apparatus, applications, or operating systems to perform operations and/or methods discussed herein. Memory 1520 can be configured to store one or more digitized stained histology slide images (e.g., obtained via optical microscopes, etc.) of tumor(s) potentially having cribriform morphology (e.g., for training and/or determining a likelihood or risk of BCR). Each of the digitized slide(s) can comprise a plurality of pixels or voxels, each pixel or voxel having an associated intensity. Memory 1520 can be further configured to store additional data involved in performing operations discussed herein, such as for constructing and/or employing a model to predict a likelihood of a biochemical recurrence (BCR) of a tumor based at least in part on a cribriform-to-tumor area ratio (CAR) determined via cribriform morphology automatically segmented via a trained deep learning model, as discussed in greater detail herein.

Apparatus 1500 can also comprise an input/output (I/O) interface 1530 (e.g., associated with one or more I/O devices), a set of circuits 1550, and an interface 1540 that connects the processor(s) 1510, the memory 1520, the I/O interface 1530, and the set of circuits 1550. I/O interface 1530 can be configured to transfer data between memory 1520, processor 1510, circuits 1550, and external devices, for example, medical imaging device(s) (e.g., optical microscopy system(s), etc.), and/or one or more remote devices for receiving inputs and/or providing outputs to a clinician, patient, etc., such as optional personalized medicine device 1560.

The processor(s) 1510 and/or one or more circuits of the set of circuits 1550 can perform one or more acts associated with a method or set of operations discussed herein, such as set of operations 100, 200, etc. In various embodiments, different acts (e.g., different operations of a set of operations) can be performed by the same or different processor(s) 1510 and/or one or more circuits of the set of circuits 1550.

Apparatus 1500 can optionally further comprise personalized medicine device 1560. Apparatus 1500 can be configured to provide the prediction of likelihood/risk of BCR and/or other data to personalized medicine device 1560. Personalized medicine device 1560 may be, for example, a computer assisted diagnosis (CADx) system or other type of personalized medicine device that can be used to facilitate monitoring and/or treatment of an associated medical condition. In some embodiments, processor(s) 1510 and/or one or more circuits of the set of circuits 1550 can be further configured to control personalized medicine device 1560 to display the automatically segmented cribriform morphology, calculated CAR, risk of BCR, and/or other data on a computer monitor, a smartphone display, a tablet display, or other displays.

Examples herein can include subject matter such as an apparatus, an optical microscopy system, a personalized medicine system, a CADx system, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system for performing any operations and/or methods discussed herein, according to embodiments and examples described.

One embodiment includes a non-transitory computer-readable storage device storing computer-executable instructions that when executed control a processor to perform operations for generating a prognosis of biochemical recurrence in invasive cribriform carcinoma (ICC), the operations comprising: accessing a digitized hematoxylin and eosin (H&E) stained image of a region of tissue demonstrating ICC, wherein the digitized H&E stained image is associated with a patient demonstrating ICC; segmenting a tumor region represented in the digitized H&E stained image, wherein the tumor region has an associated area; segmenting an ICC area represented in the digitized H&E stained image using a cribriform segmentation deep learning model trained to segment an ICC area represented in digitized H&E stained imagery; computing a cribriform-to-tumor area ratio (CAR) based on the area associated with the tumor region and the ICC area; and generating a prognosis that the patient will experience biochemical recurrence (BCR) based on the CAR and a threshold of cribriform area that maximizes separability between BCR patients and non-BCR patients.

In one embodiment, the operations further comprise generating a visual output of the prognosis, and displaying the visual output of the prognosis. Embodiments may further display the digitized H&E stained image, the tumor region, the ICC area, or the CAR. Embodiments may further display an operating parameter of cribriform segmentation deep learning model.

In one embodiment, the operations further comprise training a cribriform segmentation deep learning model to segment an ICC area represented in a digitized H&E stained image according to various techniques described herein.

Example 1 is a non-transitory computer-readable medium storing computer-executable instructions that, when executed, cause a processor to perform operations, comprising: accessing at least a portion of a digitized stained histology slide comprising a tumor; automatically segmenting, via a trained deep learning (DL) model, cribriform morphology in connection with the tumor on the at least the portion of the digitized stained histology slide; determining a cribriform-to-tumor area ratio (CAR) based at least in part on an area of the segmented cribriform morphology and an area of the tumor; and determining a risk of biochemical recurrence (BCR) of a cancer associated with the tumor based at least in part on the CAR.

Example 2 comprises the subject matter of any variation(s) of any of example(s) 1, wherein the operations further comprise post-processing the automatically segmented cribriform morphology to remove any cribriform areas smaller than a threshold area.

Example 3 comprises the subject matter of any variation(s) of any of example(s) 2, wherein the threshold area is between 0.02 mm2 and 0.03 mm².

Example 4 comprises the subject matter of any variation(s) of any of example(s) 1-3, wherein the DL model is a convolutional neural network (CNN).

Example 5 comprises the subject matter of any variation(s) of any of example(s) 4, wherein the CNN has one of a UNet architecture or a modified UNet architecture.

Example 6 comprises the subject matter of any variation(s) of any of example(s) 1-5, wherein the DL model employs one of the following optimizers: an Adam optimizer, a stochastic gradient descent (SGD) optimizer, a SGD optimizer with momentum, or a SGD optimizer with Nesterov momentum.

Example 7 comprises the subject matter of any variation(s) of any of example(s) 1-6, wherein determining the risk of biochemical recurrence (BCR) of the cancer associated with the tumor based at least in part on the CAR comprises one of: determining that there is an increased risk of BCR based on the CAR exceeding a CAR threshold, or determining that there is no increased risk of BCR based on the CAR not exceeding the CAR threshold.

Example 8 comprises the subject matter of any variation(s) of any of example(s) 7, wherein the CAR threshold is between 0.035 and 0.045.

Example 9 comprises the subject matter of any variation(s) of any of example(s) 1-8, wherein the cancer is prostate cancer.

Example 10 is a non-transitory computer-readable medium storing computer-executable instructions that, when executed, cause a processor to perform operations, comprising: accessing a training set of patches, wherein each patch is extracted from an associated digitized stained histology slide of a first plurality of digitized stained histology slides, wherein each digitized stained histology slide of the first plurality of digitized stained histology slides comprises an associated tumor, and wherein each patch of a first subset of the training set comprises cribriform morphology that has been annotated on that patch; training a deep learning (DL) model to automatically segment cribriform morphology based at least in part on the training set of patches and the annotated cribriform morphology; determining via the trained DL model, for each digitized stained histology slide of a second plurality of digitized stained histology slides, an associated cribriform-to-tumor area ratio (CAR) for that digitized stained histology slide, wherein each digitized stained histology slide of the second plurality of digitized stained histology slides is associated with a known prognosis of biochemical recurrence (BCR) or non-BCR; and determining two or more ranges of CAR values based on the associated CARs and known prognoses for the second plurality, wherein each range of CAR values of the two or more ranges of CAR values is associated with a different risk of BCR.

Example 11 comprises the subject matter of any variation(s) of any of example(s) 10, wherein the DL model is a convolutional neural network (CNN).

Example 12 comprises the subject matter of any variation(s) of any of example(s) 11, wherein the CNN has one of a UNet architecture or a modified UNet architecture.

Example 13 comprises the subject matter of any variation(s) of any of example(s) 10-12, wherein the DL model employs one of the following optimizers: an Adam optimizer, a stochastic gradient descent (SGD) optimizer, a SGD optimizer with momentum, or a SGD optimizer with Nesterov momentum.

Example 14 comprises the subject matter of any variation(s) of any of example(s) 10-13, wherein the two or more ranges of values are two ranges of values comprising a first range that is above a CAR threshold and associated with increased risk of BCR and a second range that is at or below the CAR threshold and associated with no increased risk of BCR.

Example 15 comprises the subject matter of any variation(s) of any of example(s) 14, wherein the CAR threshold is between 0.035 and 0.045.

Example 16 comprises the subject matter of any variation(s) of any of example(s) 10-15, wherein the cancer is prostate cancer.

Example 17 is an apparatus, comprising: a memory configured to store a digitized stained histology slide comprising a tumor; and one or more processors configured to perform operations comprising: automatically segmenting, via a trained deep learning (DL) model, cribriform morphology in connection with the tumor on the at least the portion of the digitized stained histology slide; determining a cribriform-to-tumor area ratio (CAR) based at least in part on an area of the segmented cribriform morphology and an area of the tumor; and determining a risk of biochemical recurrence (BCR) of a cancer associated with the tumor based at least in part on the CAR.

Example 18 comprises the subject matter of any variation(s) of any of example(s) 17, wherein the operations further comprise post-processing the automatically segmented cribriform morphology to remove any cribriform areas smaller than a threshold area.

Example 19 comprises the subject matter of any variation(s) of any of example(s) 18, wherein the threshold area is between 0.02 mm² and 0.03 mm².

Example 20 comprises the subject matter of any variation(s) of any of example(s) 17-19, wherein the DL model is a convolutional neural network (CNN).

Example 21 comprises the subject matter of any variation(s) of any of example(s) 20, wherein the CNN has one of a UNet architecture or a modified UNet architecture.

Example 22 comprises the subject matter of any variation(s) of any of example(s) 17-21, wherein the DL model employs one of the following optimizers: an Adam optimizer, a stochastic gradient descent (SGD) optimizer, a SGD optimizer with momentum, or a SGD optimizer with Nesterov momentum.

Example 23 comprises the subject matter of any variation(s) of any of example(s) 17-22, wherein determining the risk of biochemical recurrence (BCR) of the cancer associated with the tumor based at least in part on the CAR comprises one of: determining that there is an increased risk of BCR based on the CAR exceeding a CAR threshold, or determining that there is no increased risk of BCR based on the CAR not exceeding the CAR threshold.

Example 24 comprises the subject matter of any variation(s) of any of example(s) 23, wherein the CAR threshold is between 0.035 and 0.045.

Example 25 comprises the subject matter of any variation(s) of any of example(s) 17-24, wherein the cancer is prostate cancer.

Example 26 comprises an apparatus comprising means for executing any of the described operations of examples 1-25.

Example 27 comprises a computer-readable medium that stores instructions for execution by a processor to perform any of the described operations of examples 1-25.

Example 28 comprises an apparatus comprising: a memory; and one or more processors configured to: perform any of the described operations of examples 1-25.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another logic, method, or system. A circuit may include a software controlled microprocessor, a discrete logic (e.g., ASIC), an analog circuit, a digital circuit, a programmed logic device, a memory device containing instructions, and other physical devices. A circuit may include one or more gates, combinations of gates, or other circuit components. Where multiple logical circuits are described, it may be possible to incorporate the multiple logical circuits into one physical circuit. Similarly, where a single logical circuit is described, it may be possible to distribute that single logical circuit between multiple physical circuits.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A non-transitory computer-readable medium storing computer-executable instructions that, when executed, cause a processor to perform operations, comprising:
accessing at least a portion of a digitized stained histology slide comprising a tumor;
automatically segmenting, via a trained deep learning (DL) model, cribriform morphology in connection with the tumor on the at least the portion of the digitized stained histology slide;
determining a cribriform-to-tumor area ratio (CAR) based at least in part on an area of the segmented cribriform morphology and an area of the tumor; and
determining a risk of biochemical recurrence (BCR) of a cancer associated with the tumor based at least in part on the CAR.

2. The non-transitory computer-readable medium of claim 1, wherein the operations further comprise post-processing the automatically segmented cribriform morphology to remove any cribriform areas smaller than a threshold area.

3. The non-transitory computer-readable medium of claim 2, wherein the threshold area is between 0.02 mm² and 0.03 mm².

4. The non-transitory computer-readable medium of claim 1, wherein the DL model is a convolutional neural network (CNN).

5. The non-transitory computer-readable medium of claim 4, wherein the CNN has one of a UNet architecture or a modified UNet architecture.

6. The non-transitory computer-readable medium of claim 1, wherein the DL model employs one of the following optimizers: an Adam optimizer, a stochastic gradient descent (SGD) optimizer, a SGD optimizer with momentum, or a SGD optimizer with Nesterov momentum.

7. The non-transitory computer-readable medium of claim 1, wherein determining the risk of biochemical recurrence (BCR) of the cancer associated with the tumor based at least in part on the CAR comprises one of: determining that there is an increased risk of BCR based on the CAR exceeding a CAR threshold, or determining that there is no increased risk of BCR based on the CAR not exceeding the CAR threshold.

8. The non-transitory computer-readable medium of claim 7, wherein the CAR threshold is between 0.035 and 0.045.

9. The non-transitory computer-readable medium of claim 1, wherein the cancer is prostate cancer.

10. A non-transitory computer-readable medium storing computer-executable instructions that, when executed, cause a processor to perform operations, comprising:
accessing a training set of patches, wherein each patch is extracted from an associated digitized stained histology slide of a first plurality of digitized stained histology slides, wherein each digitized stained histology slide of the first plurality of digitized stained histology slides comprises an associated tumor, and wherein each patch of a first subset of the training set comprises cribriform morphology that has been annotated on that patch;
training a deep learning (DL) model to automatically segment cribriform morphology based at least in part on the training set of patches and the annotated cribriform morphology;
determining via the trained DL model, for each digitized stained histology slide of a second plurality of digitized stained histology slides, an associated cribriform-to-tumor area ratio (CAR) for that digitized stained histology slide, wherein each digitized stained histology slide of the second plurality of digitized stained histology slides is associated with a known prognosis of biochemical recurrence (BCR) or non-BCR; and
determining two or more ranges of CAR values based on the associated CARs and known prognoses for the second plurality, wherein each range of CAR values of the two or more ranges of CAR values is associated with a different risk of BCR.

11. The non-transitory computer-readable medium of claim 10, wherein the DL model is a convolutional neural network (CNN).

12. The non-transitory computer-readable medium of claim 11, wherein the CNN has one of a UNet architecture or a modified UNet architecture.

13. The non-transitory computer-readable medium of claim 10, wherein the DL model employs one of the following optimizers: an Adam optimizer, a stochastic gradient descent (SGD) optimizer, a SGD optimizer with momentum, or a SGD optimizer with Nesterov momentum.

14. The non-transitory computer-readable medium of claim 10, wherein the two or more ranges of values are two ranges of values comprising a first range that is above a CAR threshold and associated with increased risk of BCR and a second range that is at or below the CAR threshold and associated with no increased risk of BCR.

15. The non-transitory computer-readable medium of claim 14, wherein the CAR threshold is between 0.035 and 0.045.

16. The non-transitory computer-readable medium of claim 10, wherein the cancer is prostate cancer.

17. An apparatus, comprising:
a memory configured to store a digitized stained histology slide comprising a tumor; and
one or more processors configured to perform operations comprising:
automatically segmenting, via a trained deep learning (DL) model, cribriform morphology in connection with the tumor on the at least the portion of the digitized stained histology slide;
determining a cribriform-to-tumor area ratio (CAR) based at least in part on an area of the segmented cribriform morphology and an area of the tumor; and
determining a risk of biochemical recurrence (BCR) of a cancer associated with the tumor based at least in part on the CAR.

18. The apparatus of claim 17, wherein the operations further comprise post-processing the automatically segmented cribriform morphology to remove any cribriform areas smaller than a threshold area.

19. The apparatus of claim 18, wherein the threshold area is between 0.02 mm² and 0.03 mm².

20. The apparatus of claim 17, wherein the DL model is a convolutional neural network (CNN).

21. The apparatus of claim 20, wherein the CNN has one of a UNet architecture or a modified UNet architecture.

22. The apparatus of claim 17, wherein the DL model employs one of the following optimizers: an Adam optimizer, a stochastic gradient descent (SGD) optimizer, a SGD optimizer with momentum, or a SGD optimizer with Nesterov momentum.

23. The apparatus of claim 17, wherein determining the risk of biochemical recurrence (BCR) of the cancer associated with the tumor based at least in part on the CAR comprises one of: determining that there is an increased risk of BCR based on the CAR exceeding a CAR threshold, or determining that there is no increased risk of BCR based on the CAR not exceeding the CAR threshold.

24. The apparatus of claim 23, wherein the CAR threshold is between 0.035 and 0.045.

25. The apparatus of claim 17, wherein the cancer is prostate cancer.
